(19) United States
(12) Patent Application Publication
(10) Pub. No.: US 2011/0180072 A1
(43) Pub. Date: Jul. 28, 2011

(54) Assembly and method of facilitating the cleaning of an endotracheal tube

(76) Inventor: Orlando Morejon, Key Largo, FL (US)

(21) Appl. No.: 12/657,768

(22) Filed: Jan. 27, 2010

(51) Int. Cl. B08B 9/04; A61M 16/04

(52) U.S. Cl. 128/207.14

(57) ABSTRACT

A handle assembly and method of its use which facilitates the cleaning of the interior of an endotracheal tube. The handle assembly comprises a body structured to be held and manipulated by a hand of a user and is structured to removably retain an inflation device. A tubular member is connected to the body and is dimensioned to be inserted within and pass along an interior length of the endotracheal tube to facilitate the cleaning thereof. A calibration assembly is movably connected to the body and structured to determine an accurate or appropriate length of the tubular member to be inserted within the endotracheal tube to avoid over insertion of the tubular member. A positioning member associated with the calibration assembly is connected to the restricting portion and is movable along the length of the body to position the positioning/stop member adjacent the proximal end of the endotracheal tube for alignment and determination of the accurate length of the tubular member to be inserted.

### BACKGROUND OF THE INVENTION

1. Field of the Invention

This invention is directed to a handle assembly and a method of using it to facilitate the cleaning of an interior of an endotracheal tube. A hand held body comprises a calibration assembly which determines the proper length of a tubular member/catheter to be inserted within the endotracheal tube to efficiently accomplish the cleaning procedure. The calibration assembly is manipulated for selective positioning on and relative to the body in order to determine an appropriate length of the tubular member to be inserted within the endotracheal tube to accomplish the cleaning procedure, while preventing the potentially dangerous extension of the tubular member out of the open internal, distal end of the endotracheal tube.

2. Description of the Related Art

Many patients in a hospital and in particular, patients in an Intensive Care Unit (“ICU”) must be fitted with endotracheal tubes to facilitate their respiration. Specifically, an endotracheal tube is an elongate, semi-rigid lumen which is inserted into a patient's nose or throat and projects down into airflow communication with the patient's respiratory system. As such, the patient either directly, or with the aid of a respiratory unit, is able to breathe more effectively through the endotracheal tube.

Recent studies have determined, however, that the accumulation of dried tracheo-bronchial secretions on the interior wall surface of an operating endotracheal tube effectively decreases the lumen cross section, and thereby significantly increases the work of breathing for the intubated patient. Moreover, increasing the work of breathing for the patient necessitates that a higher level of support be provided to compensate and often results in the patient's incubation period and ICU stay being significantly prolonged. Furthermore, it is also seen that thick secretions on the walls of the endotracheal tube often serve as a nidus for continued infection in the lungs, leading to added morbidity and hospital costs for the intubated patient.

To date, the only effective means of eliminating the accumulated secretions within an endotracheal tube has been to exchange the contaminated endotracheal tube for a new tube. However, there are several disadvantages to this procedure, such as temporary arrest of ventilatory support and the risk of complete loss of airway control. For example, re-incubation may be exceedingly difficult in patients with swelling of the soft tissue of the neck, and in patients having cervical spine immobilization. More specifically, upon removal of the endotracheal tube, the appropriate internal passages tend to close up or be otherwise difficult to isolate for reintroduction of a new endotracheal tube. Further, re-intubation of a patient can result in additional trauma to the oral, laryngeal and tracheal tissues.

Short of replacing the endotracheal tube, other procedures currently in use for maintaining a clean endotracheal tube include the use of flexible suction/irrigation catheters. Specifically, a suction/irrigation catheter is passed down the endotracheal tube and upper airways in an attempt to evacuate contaminants from the lumen. Unfortunately, although the suction/irrigation catheters generally clear the airway of watery secretions, they are ineffective at clearing the inspissated secretions that have accumulated on the inner wall surface of the endotracheal tube over the course of days. In essence, the use of a suction/irrigation catheter merely delays the inevitable, namely, that the endotracheal tube be removed and replaced.

One somewhat recent attempt to address the problems associated with the maintenance of endotracheal tubes is seen to provide a two part assembly which is introduced into the flow through passage of the endotracheal tube. Specifically, a thin interior, solid segment having a plurality of retracting bristles and a sealing gasket at an end thereof is contained within an exterior lumen. In use, the entire coupled assembly is introduced into the endotracheal tube, and an interior segment is pushed through an outer tube so that the bristles expand to engage the wall surface. In addition, a gasket member, such as a foam cylinder or balloon, expands to completely seal off the area behind the bristles. The entire device, including the upwardly angled bristles is then pulled upwardly with the gasket element completely sealing off the tube there below so that any debris removed by the bristles is retained.

Such a device, however, does not provide for the indication of an accurate insertion in order to prevent over-insertion into the endotracheal tube. Furthermore, it is seen from the need to include the bristles, the direct engagement of a gasket type member, such as the balloon, with the interior wall surface of the endotracheal tube, does not provide for the complete and effective removal of secretions, due primarily to the smooth exterior surface of the gasket. Moreover, the smooth resilient material surface also results in substantial friction between the rubbery gasket and the plastic wall surface, thereby making it quite difficult to smoothly and effectively pull the cleansing device from the endotracheal tube. Additionally, it is recognized that the upwardly angled bristle members are susceptible to complete or partial retraction as they encounter obstacles in an attempt to scrape clean the interior of the endotracheal tube. In fact, the bristle members are often quite sharp and may be damaging to the endotracheal tube or to a patient if inadvertently projected beyond the open interior, distal end of the endotracheal tube allowing for the possibility of the outwardly projecting bristle members becoming stuck outside the endotracheal tube. Also, because of the collapsing configuration of bristles, gaps will naturally exist between adjacent bristles resulting in some areas of the tube not being engaged. Accordingly, as secretions begin to build up beneath the bristles, their collapse is further restricted. Further, such a single function device necessitates that additional items be introduced into the tube, generally resulting in additional trauma to the patient, such as when suction becomes necessary.

As such, there is still a substantial need in this art for a cleaning assembly that can be used to clear endotracheal tube secretions effectively, and on a regular basis, thereby expediting ventilatory weaning and extubation of ICU patients. Further, there is a need for an effective endotracheal tube cleaning apparatus which can be easily and effectively introduced into the endotracheal tube. Such a cleaning assembly should be easily removed while effectively removing solid secretion buildup, due at least in part to its friction minimizing engagement with the interior wall surface of the endotracheal tube and/or because of its alleviation of negative pressure/suction within the endotracheal tube upon removal thereof. Additionally, there is a need for a cleaning assembly which can be accurately extended into the endotracheal tube without substantial risk of over introduction.

In addition to the referenced needs in the industry, it is also noted that an effective cleaning assembly should preferably be structured to facilitate the operative use of cooperative devices which facilitate the cleaning procedure. Moreover, the cleaning assembly should maintain maximum sterile integrity as to those components which will be used to provide an effective cleaning procedure.

### SUMMARY OF THE INVENTION

The present invention is directed to a handle assembly operatively structured to be used with cooperative components which collectively, when the handle assembly is assembled therewith, will facilitate the efficient and effective cleaning of an interior of the endotracheal tube. As will be explained in greater detail hereinafter, the aforementioned cooperative components used with the handle assembly may include an expansion device and a tubular member which may be in the form of a cleaning catheter. The expansion device may comprise inflation device such as, but not limited to, a syringe type structure operative to inject a predetermined quantity of air into an inflatable, expandable cleaning assembly associated with the tubular member/cleaning catheter. Moreover, the tubular member/catheter assembly and the cleaning assembly associated therewith may be of the type disclosed in U.S. Pat. Nos. 5,709,691; 6,082,361; 6,494,208; 6,679,262; and 7,060,135, all issued to the inventor of the present invention.

As such, the elongated tubular member and/or cleaning catheter has a transverse dimension sufficiently less than the interior of the endotracheal tube to facilitate its insertion therein. Further, the length of the tubular member is such as to extend substantially along an entire length of the endotracheal tube. The tubular member includes a distal end and a proximal end, wherein the aforementioned cleaning assembly is disposed adjacent to the distal end. The cleaning assembly is structured to be expanded or inflated, by the aforementioned expansion device or inflation syringe, and thereby be expanded radially outward from the tubular member into a cleaning orientation. When so expanded or inflated, the cleaning assembly will confront the interior surfaces of the endotracheal tube in a manner which facilitates the removal of any secretions or other material collected thereon. Moreover, the various embodiments of the cleaning assembly include an outer peripheral surface having at least a portion thereof comprising an irregular configuration structured to facilitate a cleaning force being exerted on the interior surface of the endotracheal tube when the cleaning assembly is in the inflated, cleaning orientation. As a result, the withdrawal of the tubular member, while the cleaning assembly is expanded, will cause the cleaning assembly to move from the distal or inner end of the endotracheal tube to and be withdrawn from the outer open end of the endotracheal tube while collecting and causing the removal of any secretions or other material collected within the endotracheal tube.

Accordingly, the handle assembly of the present invention is structured to facilitate the cleaning of the endotracheal tube by assuring that an accurate or predetermined length of the tubular member is inserted into the interior of the endotracheal tube and prevent an “over insertion”. Such an over insertion could result in an undesirable extension or protrusion of the distal end of the tubular member and/or the cleaning assembly from protruding outwardly from the distal open end of the endotracheal tube. As such, once the proper length of the tubular member is determined, it is inserted into the endotracheal tube resulting in the cleaning assembly being disposed in a proper position for expansion into its cleaning orientation. As set forth above, such expansion inflation brings the outer surface of the cleaning assembly into confronting relation with the interior surface of the endotracheal tube and effectively facilitates performance of the cleaning procedure. It is emphasized that the expansion device may be other than an inflation device or inflation syringe as set forth above. Such a non-inflation expansion device should be cooperatively functional with an appropriate cleaning assembly to facilitate the selective expansion of the cleaning assembly into the cleaning orientation.

Accordingly, the handle assembly of the present invention comprises a body structured to be held by one hand of a user in a manner which allows for the manipulation of the inflation device as well as other components associated with or connected to the body of the handle assembly. Moreover, the body includes a proximal end and a distal end, wherein the elongated tubular member is connected to the distal end. An appropriate connection of the tubular member to the distal end of the body accomplishes fluid communication between the inflation device and the cleaning assembly through at least a portion of the interior of the tubular member. Such fluid communication provides for selective inflation or radial expansion of the cleaning assembly when it is accurately positioned within the interior of the endotracheal tube.

In addition to the above, the handle assembly includes a calibration assembly movably connected to the body and structured to determine the proper and/or accurate length of the tubular member to be inserted within the endotracheal tube in order to prevent over insertion as generally set forth above. The accurate or proper length of the tubular member being inserted within the endotracheal tube will also automatically dispose the aforementioned cleaning assembly in a proper position to maximize cleaning thereof, when inflated into the cleaning orientation.

More specifically, the calibration assembly includes a restricting portion or stop member which is also accurately defined and referred to herein as a calibrator disk. The stop member may assume a variety of different configurations and/or structures, which facilitate the alignment and/or confronting relation of the restricting portion or stop member with the outer or proximal end of the endotracheal tube, while it is inserted within the patient. The calibration assembly further comprises a positioning member which may also be accurately referred to herein as a “depth calibrator tab” movably connected to the body of the handle assembly. This positioning member is exteriorly accessible so as to be engaged and selectively positioned along the length of the body by being engaged and/or manipulated a finger of the user. The positioning member or depth calibrator tab is fixedly interconnected to the restricting portion or stop member such that movement of the positioning member/depth calibrator tab will serve to concurrently move the positioning portion/stop member outwardly from the distal end of the body.

As such, manipulation of the positioning member will serve to efficiently dispose the restricting portion or stop member into aligned relation and/or confronting engagement with the extremity of the proximal end of the endotracheal tube. Further, the manipulation of the positioning member will cause the stop member to move along the length of the tubular member once the tubular member is connected to the distal end of the body. As more specifically described, the cooperative structuring and operation of the calibration assembly and the tubular member will thereby provide an efficient and reliable way of determining the accurate, predetermined length of the tubular member to be safely inserted within the endotracheal tube in order to properly position the cleaning assembly therein, prior to inflation. Such determination of the inserted length of the tubular member will also prevent the distal end of the tubular member, or cleaning assembly associated therewith, from extending outwardly from the open end of the endotracheal tube.

Other structural and operative features of the handle assembly include the body having a suction port. The suction port is disposed and structured to removably connect at least a portion of the interior of the tubular member to an appropriate vacuum source, thereby allowing for the removal of watery secretions and other material from the interior of the endotracheal tube by suction. An appropriate opening or access port is disposed or formed in the distal end of the tubular member preferably, but not exclusively, beyond the location of the cleaning assembly to further aid in this removal by suction.

As also explained in greater detail hereinafter, the method of use of the handle assembly of the present invention which facilitates the cleaning of the endotracheal tube includes the establishing of a predetermined alignment between the tubular member/cleaning catheter and an exterior of the endotracheal tube. Such alignment is accomplished by using appropriate indicia, such as numerical or calibration markers, present on both the endotracheal tube and tubular member. More specifically, by selectively moving the positioning member/calibrator tab along the length of the body of the handle assembly the restricting portion or stop member will be extended outwardly from the distal end of the body. Such manipulation of the calibration assembly is accomplished while maintaining alignment between the numbered or calibration markers of the indicia on both the tubular member and the exterior of the endotracheal tube.

The aforementioned manipulation of the calibration assembly serves to dispose the positioning portion or stop member into aligned and/or confronting relation with the exterior extremity of the proximal, open end of the endotracheal tube, while maintaining alignment between the calibration markings on the tubular member and the endotracheal tube. The stop member will thereby be removably but securely disposed along the tubular member. Once the stop member is so disposed along the tubular member, the tubular member will be inserted through the open proximal end of the endotracheal tube until the restricting portion or stop member is disposed in confronting relation with the extremity of the open end of the endotracheal tube. As a result, no additional length of the tubular member can be inserted into the endotracheal tube to the confronting engagement of the stop member with the extremity of the outer open end of the endotracheal tube.

Thereafter, the inflation device removably retained on or within the body of the handle assembly is manipulated so as to inflate and thereby expand the cleaning assembly into its expanded, cleaning orientation, causing the outer surface thereof to confront the interior surface of the endotracheal tube. While maintaining the cleaning assembly in the expanded, cleaning orientation, the tubular member is withdrawn from the endotracheal tube thereby serving to collect and remove any secretions or other material from the interior of the endotracheal tube.

Accordingly, the structure and method of use of the handle assembly of the present invention, when assembled with the aforementioned operative components, will serve to efficiently and reliably clean the interior of the endotracheal tube while it remains in an operative position within a patient.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a top perspective view of a handle assembly of the present invention structured to facilitate the retention and operation of an operative components utilized in the cleaning of an interior of an endotracheal tube.

FIG. 2 is a perspective view of the embodiment of FIG. 1 assembled with an inflation device and a tubular member/cleaning catheter.

FIG. 3 is a side perspective view of the embodiment of FIG. 2 including a calibration assembly.

FIG. 4 is a front view of the embodiment of FIGS. 2 and 3.

FIG. 5 is a side view of the embodiment of FIGS. 2-4.

FIG. 6 is a rear view of the embodiment of FIGS. 2-5.

FIG. 7 is a side view opposite to that of FIG. 5 of the embodiment of FIGS. 2-6.

FIG. 8 is an end view of the embodiment of FIGS. 2-7.

FIG. 9A is a perspective view in partial cutaway of another preferred embodiment of the handle assembly of the present invention including a calibration assembly differing from the calibration assembly represented in FIGS. 1, 3-5, 7 and 8.

FIG. 9B is a detailed view in partial cutaway of portions of the embodiment of FIG. 9A.

FIGS. 10A and 10B are schematic representations in partial cutaway and section representing different embodiments of a fluid communication connection between the tubular member/catheter and the body of the handle assembly of the embodiment of FIGS. 1-8.

FIGS. 11A and 11B are rear and front views respectively of an assembled handle assembly as represented in FIGS. 1-8 further including labeled indicators of the structural and operative features of the assembled embodiments of the handle assembly.

FIG. 12 is a front view in partial cutaway representing operative steps in the method of use of the embodiment of FIGS. 1-8, 11A and 11B.

FIG. 13 is a front view in partial cutaway of the embodiment of FIG. 12 representing additional steps in the method of utilization of the handle assembly of the present invention.

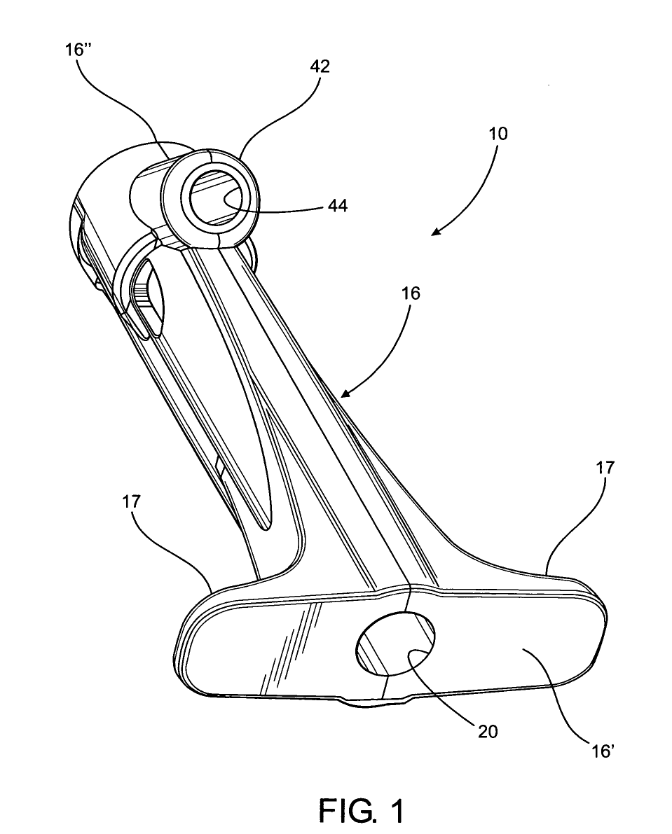

Like reference numerals refer to like parts throughout the several views of the drawings.

### DETAILED DESCRIPTION OF THE PREFERRED EMBODIMENT

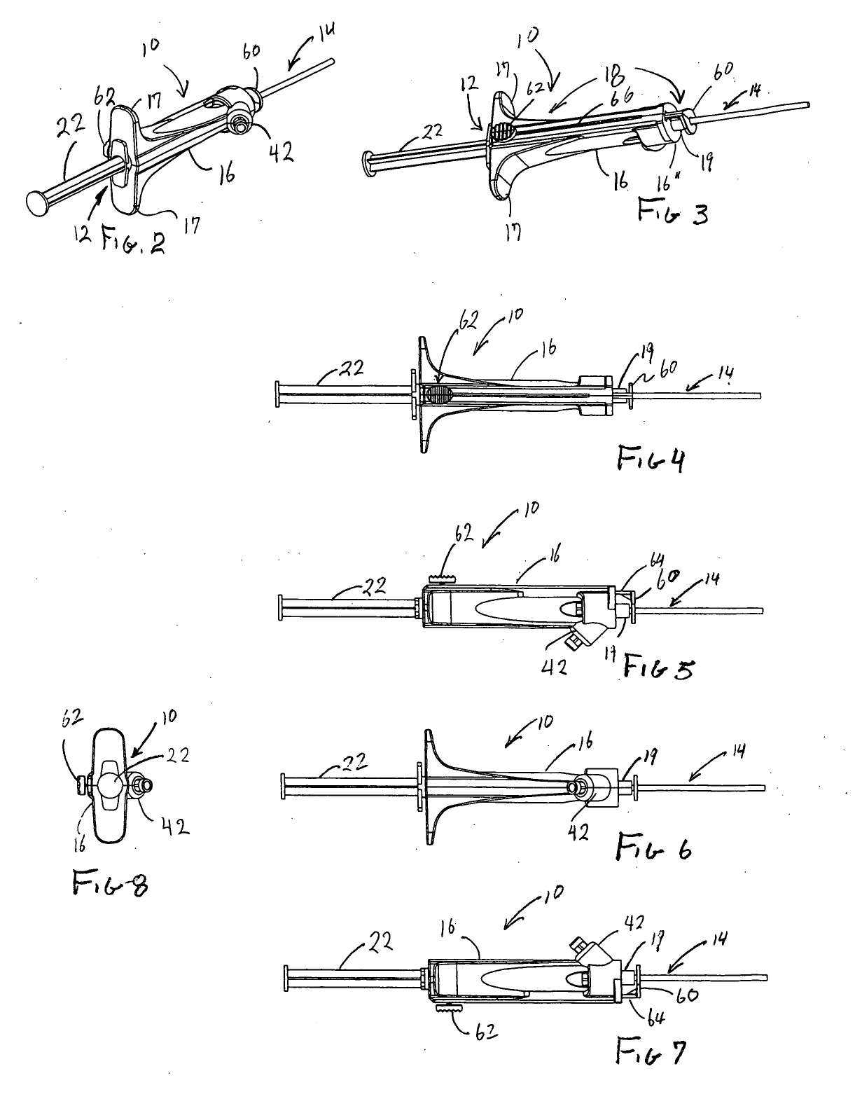

As shown in the accompanying drawings, the present invention is directed to a handle assembly, generally indicated as 10, which when connected to or assembled with other operative components, facilitates the cleaning of an interior of an endotracheal tube 100, while the endotracheal tube is positioned within a patient. As set forth in greater detail hereinafter, the aforementioned cooperative components with which the handle assembly 10 is assembled and/or connected includes an expansion device generally indicated as 12 and an elongated tubular member or catheter structure 14 represented in an assembled form in FIGS. 2-8, 9A, 9B, 11A, 11B, 12 and 13.

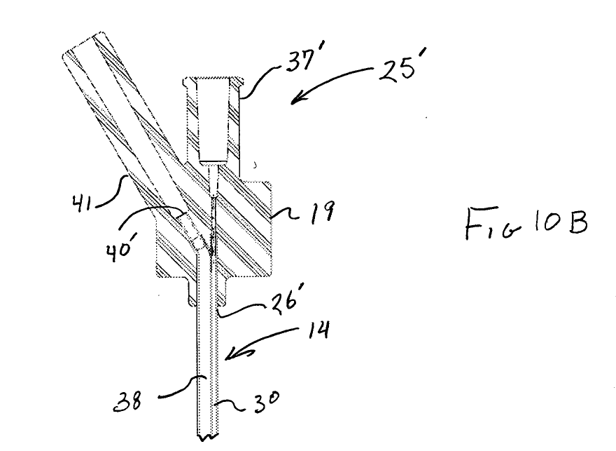

The handle assembly 10 comprises a body generally indicated as 16 having a generally elongated configuration and being further structured and configured to be held by a hand of a user, as at least partially represented in FIG. 13. As represented the body 16 may be gripped in a manner which facilitates the manipulation of the handle assembly 10, expansion device 12 and tubular member 14 as well as a calibration assembly 18 movably connected to the body 16. While the specific configuration of the body 16 may vary, it is shaped to accommodate the removable retention of the expansion device 12 on the interior thereof such as through an access opening 20. The expansion device 12, as represented throughout the accompanying Figures is preferably in the form of an inflation device such as, but not limited to, an inflation syringe. As such, the manipulation of a plunger 22 of the inflation syringe 12 will serve to draw air into the interior of the barrel (not shown) of the syringe 12 and thereafter selectively force the contained air from the barrel into and through the tubular member 14, so as to expand a cleaning assembly 24. Accordingly, the cleaning assembly 24 may be of the type represented in FIG. 10A and also of the type disclosed and described in at least some of the embodiments of U.S. Pat. Nos. 5,709,691; 6,082,361; 6,494,208; 6,679,262; 7,060,135, all issued to the inventor of the present invention herein.

The gripping of the handle 16 is further facilitated through the provision of finger engaging or finger supporting wings 17. Therefore, the body 16 may be gripped in a manner which also allows for the efficient, concurrent manipulation of the plunger 22 resulting in the inflation and expansion of the cleaning assembly 24 in the outwardly extended, cleaning orientation as represented in FIG. 13. The proximal end 16′ of the body 16 is adjacent to the wings 17 and at least partially accommodates the placement of the inflation syringe 12. The tubular member or cleaning catheter 14 is connected to the opposite or distal end 16″ as at connecting hub 19. With further reference to FIG. 10A, the connecting hub 19 is at least generally associated with a connection assembly generally indicated as 25 associated with the proximal end 26 of the tubular member 14. Therefore, the tubular member 14 and in particular the connecting assembly 25 is structured to establish fluid communication with the inflation device and/or syringe 12 once it is operatively connected to and retained within the interior of the body 16 as represented in FIGS. 2-8. More specifically, the tubular member 14 includes at least one interior, elongated, tubular passage 30 having an outlet 32 communicating with the interior of an inflatable balloon or similar structure 33 associated with the cleaning assembly 24. As a result, the forcing of air initially disposed within the barrel of the inflation syringe 12 will serve to inflate and thereby radially expand the cleaning assembly 24. Upon expansion, the exterior surface 35 will engage or otherwise substantially confront the interior surface of the endotracheal tube in a manner which will facilitate the removal of secretions collected within the endotracheal tube 100. Accordingly, FIG. 10A represents an inflated, radially expanded cleaning orientation of the cleaning assembly 24, which is accomplished by the depression of the plunger 22 once the inflation device or syringe 12 has initially been at least partially filled with an appropriate quantity of air necessary to inflate the cleaning assembly 24 into its cleaning orientation.

Interconnection between the passage 30 and the inflation device or syringe 12 may be accomplished by an appropriate tubing or conduit type connector 37 connected to the dispensing end of the inflation syringe 12. As also represented in FIGS. 10A and 10B, the tubular member or cleaning catheter 14 also includes an additional channel 38 disposed therein and extending along the length thereof. The proximal end of the channel 38 is connected, as at 40, to a suction port 42 having an access opening or connecting port 44. As such, the suction port 42 and associated connecting port 44 is removably connected to a vacuum source. Moreover, the vacuum source, once activated, will cause a negative pressure to exist within the interior of the channel 38 of sufficient strength to provide the removal by suctioning, schematically indicated by the directional arrows 50, of any secretions or other material. Therefore, the interior or distal end 52 of the tube 14 will include an entrance 54. The collected material to be removed is suctioned as at 50 through the entrance 54 and pass along the length and the interior of the channel 38 and tubular member 14, into and through the suction port 42.

With primary reference to FIG. 10B, a structural modification of the connecting assembly is generally represented as 25′ and is structured to establish fluid communication between the expansion device or inflation syringe 12 and the cleaning assembly 24. More specifically, interconnection between the passage 30 and the expansion device or inflation syringe 12 may be accomplished by a conduit 37′, appropriately connected to the dispensing end of the inflation syringe 12. As also represented, the tubular member or cleaning catheter 14 includes the additional channel 38 extending along the length thereof. The proximal end of the channel 38 is connected, as at 40′, to the suction port 42 by means of conduit 41. As set forth above, the vacuum source (not shown) once activated, will cause a negative pressure to exist within the interior of the channel 38 of sufficient strength to provide the removal or suctioning, schematically represented in FIG. 10A as directional arrows 50, of any secretions or other material collected on the interior or the endotracheal tube 100. As also represented in FIGS. 10A and 10B, the connecting hub 19 is at least generally associated with the connection assembly 25 and 25′ associated with the proximal end 26 and 26′ of the tubular member 14. Therefore, the tubular member 14 and in particular the fluid flow connecting assemblies 25 and 25′ are structured to establish fluid communication with the expansion device or inflation syringe 12 once it is operatively connected to and retained within the interior of the body 16, as represented in FIGS. 2-8.

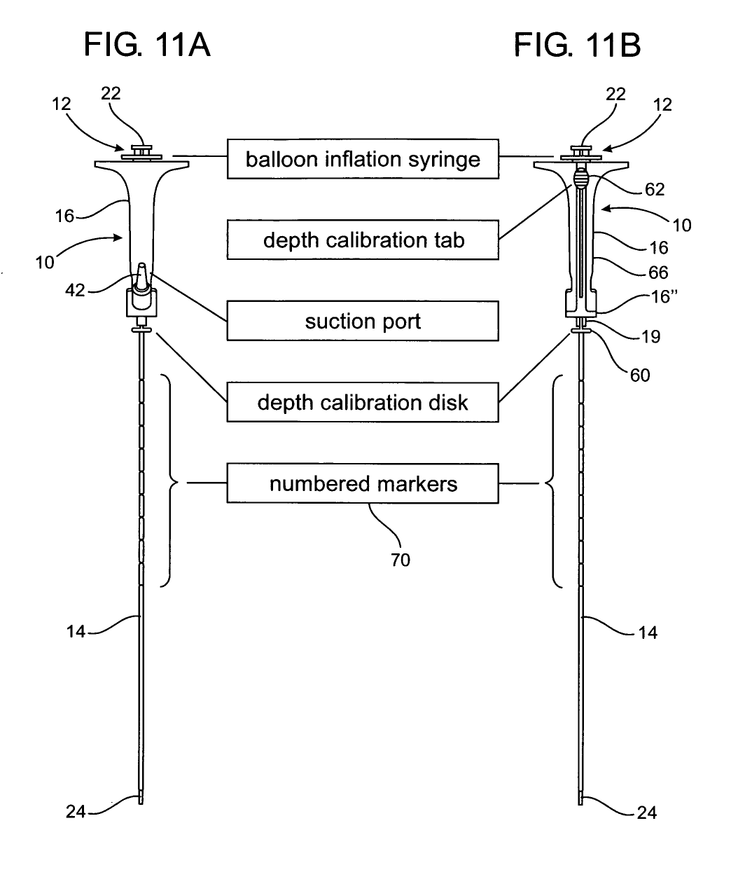

Yet another structural and operative feature of the present invention comprises a calibration assembly 18 the structure of which is represented in FIGS. 2-8 and the operation of which is primarily represented in FIGS. 11-13. More specifically, the calibration assembly 18 comprises a restricting portion or stop member 60 movably connected to or mounted on the body 16. The restricting portion or stop member 60 is interconnected to a positioning member 62 also movably connected to and/or mounted on the body 16. More specifically, the positioning member 62 comprises an exteriorly accessible finger engaging tab, which also may be accurately referred to as a depth calibrator tab. Interconnection between the restricting portion or stop member 60 and the positioning member or calibrator tab 62 is accomplished by a fixed, elongated connecting link 64 fixedly interconnecting the stop member 60 and the positioning member 62.

Accordingly, selective movement of the positioning member 62, along the length of the body 16 within the accommodating, elongated retaining slot 66, serves to concurrently move the stop member 60 relative to the body 16 and outwardly from the distal end 16″ of the body and the connecting hub 19. Further, the stop member 60 is cooperatively disposed and structured with the elongated tubular member 14, to at least partially define a movable, retaining engagement and/or relation therewith, once it is connected to the body 16. In at least one embodiment, the stop member 60 includes a disk like structure having a central opening or aperture through which the tubular member 14 is disposed and retained, while allowing movement of the stop member along a variable length of the tubular member. Therefore, the stop member 60 moves and/or is variably positioned relative to and exteriorly of the body 16 concurrently to manipulation and selective positioning of the calibrator tab or positioning member 62, along a length of the body 16. Further, in that the stop member 60 and the positioning member 62 are fixedly connected to one another by the link 63 they will move or travel a common distance. Also, in that the positioning member 62 may be disposed at any one of a plurality of locations along the length of the slot 66 and the stop member 60 moves with the positioning member 62 a common distance, both the stop member 60 and the positioning member 62 can accurately be described as being variably positioned or moved a variable distance relative to the housing 16 and/or tubular member 14.

Additional structural features include a plurality of serrations, notches or other appropriate structure lining or directly associated with the retaining slot 66. Such serrated or notched structuring may extend along a majority or substantially the entire length of the edges, peripheral portions or other appropriate parts of the retaining slot 66. In addition, such serrated or notched structuring is disposed and cooperatively structured with the positioning member or calibrator tab 62 so as to facilitate a removable but securely locked positioning of the positioning member or calibrator tab 62 at any point along the length of the body 16 and the retaining slot 66. By way of example only, a biased or other appropriately structured retaining mechanism may be directly associated with the calibrator tab 62. Such a retaining structure forces a portion of the calibrator tab 62 into the aforementioned removably locked position within the slot 66. Moreover, such a retaining mechanism may be further structured to disengage the positioning member 62 from the slot 66 by the exertion of a pressing force or other directed force on the positioning member 62, such as by the finger of a user. This will disengage the positioning member 62 from the slot 66 and allow the positioning member 62 to be moved to any appropriate location along the length of the body 16 and retaining slot 66. Such movement of the positioning member 62 relative to the body 16 and slot 66 will cause a concurrent movement of the restricting portion or stop member 60 outwardly or inwardly relative to the distal end 16″ of the body 16 and along the length of the tubular member 14. As set forth above the distance “traveled” by the positioning member 62 along the length of the body 16 and retaining slot 66 will be equal to the distance of the travel of the restricting portion or stop member 60 relative to the tubular member 14, due to the fixed interconnection of the link 64 between the positioning member or calibrator tab 62 and the stop member 60.

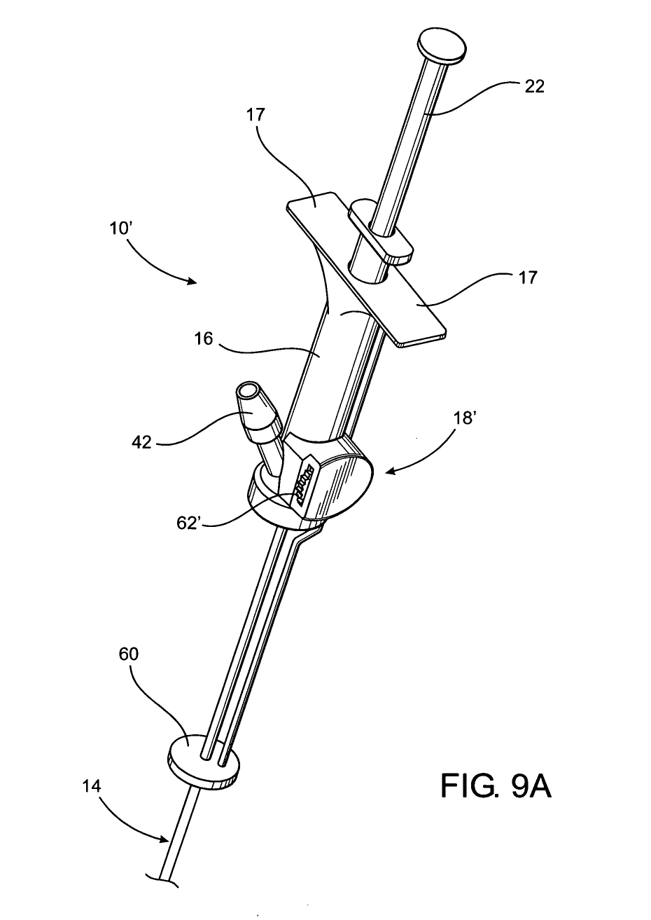

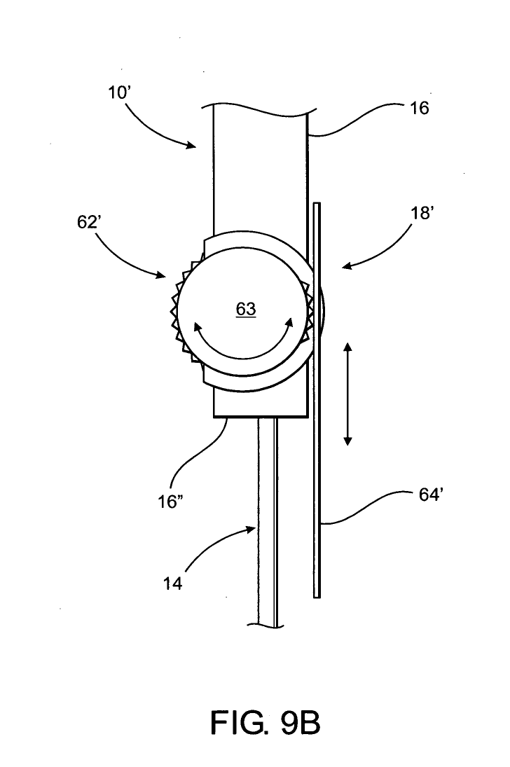

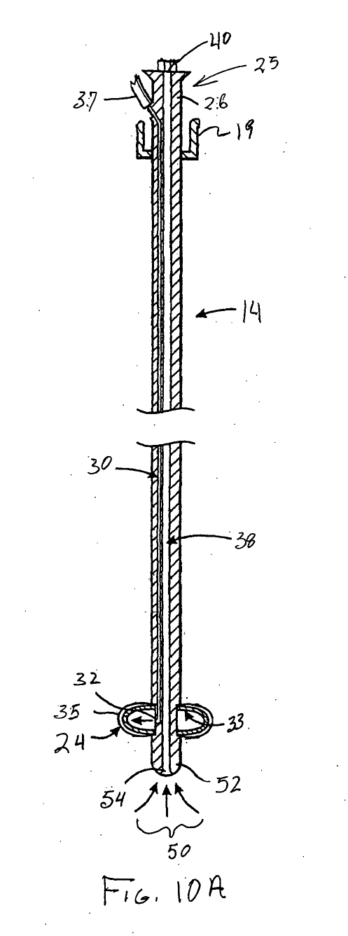

Yet another preferred embodiment of the calibration assembly is generally represented as 18′ in FIGS. 9A and 9B. Unlike the calibration assembly 18 in FIGS. 2-8, the stop member 60 is disposed outwardly from the distal end of the body 16 through manipulation of a positioning member 62′, in the form of a finger activated or manipulated wheel 63, rotationally connected to the body 16, as represented in detail in FIG. 9B. Further, the exterior peripheral surface of the wheel 63 may include gear teeth or may be knurled, roughened or otherwise structured to facilitate driving engagement with at least a portion of the length of the connecting link 64′. As with the connecting link 64 in the embodiment of the calibration assembly 18, represented in FIGS. 2-8, the connecting link 64′ is also fixedly connected to and movable with the stop member 60. Accordingly, rotation of the positioning member 62′, in the form of the calibration wheel 63, will serve to move the connecting link 64′ and the stop member 60 either towards or away from the distal end 16″ of the body 16.

The method of use of the handle assembly 10 or 10′ with regard to its cleaning of the endotracheal tube 100 is represented in FIGS. 11-13 comprises manipulation of the calibration assembly 18 or 18′ to the extent of accomplishing a predetermined alignment between the tubular member 14 and the endotracheal 100. As represented in the indicated Figures, the tubular member 14 may typically have appropriate indicia such as a plurality of numbered markers collectively indicated as 70. Moreover, markers 70 may at least partially correspond in position, dimension and/or configuration to corresponding indicia, also in the form of numbered markers 72, disposed on or visible from the exterior of the endotracheal tube 100. Once the indicia 70 on the tubular member 14 and the indicia 72 on the endotracheal tube 100 are aligned as represented in FIG. 11, the stop member 60 is extended outwardly from the distal end 16″ of the body 16 by manipulation of the positioning member or depth calibrator tab 62 along the length of the body 16. Further, the stop member (calibrator disk) 60 is extended outwardly until it becomes aligned with the extremity or outermost end 100′ of the endotracheal tube 100. The equal distance of travel or movement of the positioning member/calibrator tab 62 and the stop member 60 are indicated by correspondingly disposed directional arrows in FIG. 11. The disposition of the stop member 60 in aligned relation with the extremity or outermost end 100′ of the endotracheal tube 100 as represented in FIG. 11 serves to determine or establish the proper and accurate length of the tubular member/cleaning catheter 14 which will be inserted into the interior of the endotracheal tube 100. After such length 14′ of the tubular member 14 has been established and subsequent to the alignment between the tubular member 14 and the endotracheal tube 100 through the aligned relation of the indicia 70, 72, the determined length 14′ of the tubular member 14 can then be inserted into the endotracheal tube 100 as represented in FIG. 13. Maintenance of the stop member or calibrator disk 60 in the appropriate position to maintain and establish the accurate length 14′ of the tubular member 14 is accomplished by the aforementioned removably, but stable, locked engagement of the positioning member or calibrator tab 62 within the retaining slot 66 due to interlocking engagement as set forth above. Again, FIG. 13 is a schematic representation of the stop member 60 confrontingly engaging the outer end or extremity 100′ accurately and reliably such that the indicia 70 and 72 remain aligned.

It should be further noted that one additional advantage of positioning the stop member 60 relative to the outer end 100′ of the endotracheal tube 100, exclusively by the manipulation of the positioning member or calibration tab 62, is the avoidance of contamination of the endotracheal tube 100. More specifically, the alignment or engagement of the stop member 60 with the end 100′ of the endotracheal 100 is accomplished only by moving or positioning the calibration tab 62. Accordingly a user deliberately touching or inadvertently contacting the open, outer end 100′ of the endotracheal tube 100 is eliminated or significantly restricted.

The actual insertion and operative positioning of the appropriate length 14′ of the tubular member 14 within the endotracheal tube 100 is schematically represented in FIG. 13 and is accomplished by gripping the endotracheal tube 100 with one hand of the user, a spaced distance from the open, outer 100′ and gripping and manipulating the handle assembly 10, including body 16, with the other hand of the user. When the portion 14′ of the tubular member 14 is inserted within the endotracheal tube 100, the cleaning assembly 24 will be properly disposed within the tube 100. The plunger 22 may then be depressed as indicated by the associated directional arrow so as to inflate the cleaning assembly 24 into the cleaning orientation as explained with primary reference to FIG. 10A.

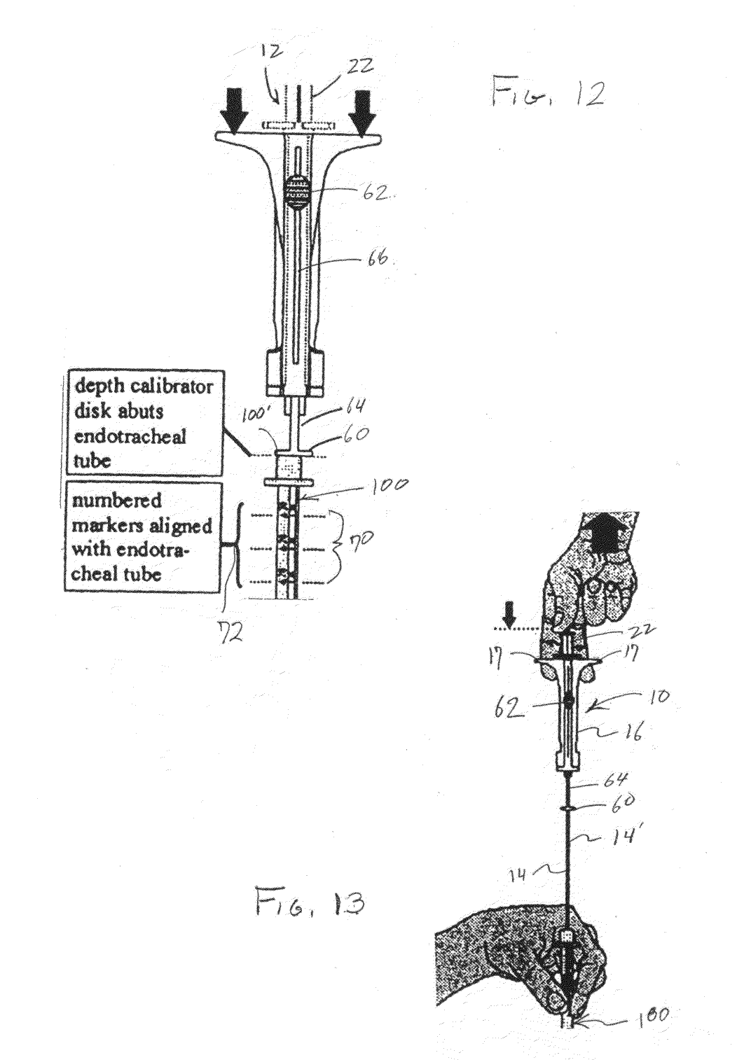

As set forth above, the endotracheal tube 100 may typically include appropriate indicia 72 such as, but not limited to the numbered markers represented in FIG. 12. However, use of the handle assembly 10 and/or 10′ may be used to clean endotracheal tubes 100 absent such indicia 72. This can be accomplished by calibrating the tubular member or cleaning catheter 14 by alignment with the appropriate indicia 72 located on an endotracheal tube which is not operatively positioned within a patient. Such a supplementary or auxiliary endotracheal tube (not shown), while not disposed within the patient, should be of the same make, model number, size, etc. that is operatively positioned within the patient, but which includes appropriate indicia 72. Accordingly, alignment of the indicia 70 and 72 appearing on the tubular member 14 and supplementary endotracheal tube, respectively, which is not located within the patient, can accomplish proper calibration in the manner set forth above as described with specific reference to FIG. 12.

Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

Now that the invention has been described,

What is claimed is:

1. A handle assembly structured to facilitate cleaning of an interior of an endotracheal tube, said handle assembly comprising:
a body including a proximal end and a distal end, said body being configured for gripping by a hand of a user,
said body structured for connection of said distal end to an elongated tubular member,
whereby the tubular member is cooperatively dimensioned with the endotracheal tube to facilitate insertion of the tubular member within and along a length of the endotracheal tube,
a calibration assembly movably connected to said body and structured to determine an appropriate length of the tubular member to be inserted within the endotracheal tube, and
said calibration assembly including a restricting portion operatively disposed relative said body and the tubular member to restrict an excessive length of the tubular member being inserted into the endotracheal tube.

2. A handle assembly as recited in claim 1 wherein said restricting portion is movable relative to said body along a length of the tubular member.

3. A handle assembly as recited in claim 2 wherein said restricting portion is extendable a variable distance from said distal end along the length of the tubular member.

4. A handle assembly as recited in claim 2 wherein said restricting portion is movable exteriorly of said distal end in movable engagement with the tubular member.

5. A handle assembly as recited in claim 2 wherein said restricting portion comprises a stop member extendable outwardly from said distal end in at least partially retaining engagement with the tubular member.

6. A handle assembly as recited in claim 1 wherein said body further comprises an interior dimensioned and configured to receive an expansion device therein.

7. A handle assembly as recited in claim 6 wherein said interior is structured to receive an inflation syringe therein.

8. A handle assembly as recited in claim 7 wherein said interior is structured to removably retain the inflation syringe within said body.

9. A handle assembly as recited in claim 7 wherein the tubular member extends outwardly from said distal end and is disposed in fluid communicating relation with the inflation syringe.

10. A handle assembly as recited in claim 1 wherein said body further comprises a vacuum port disposed in fluid communication with the tubular member.

11. A handle assembly as recited in claim 1 wherein said restricting portion comprises a stop member extendable outwardly from said body along a variable length of said tubular member; said calibration assembly further comprising a positioning member connected to said stop member and concurrently movable therewith relative to said body.

12. A handle assembly as recited in claim 11 wherein said positioning member comprises a calibrator tab disposed in an accessible position at least partially on an exterior of said housing and structured to be engaged by a finger of a user.

13. A handle assembly as recited in claim 11 wherein said positioning member is disposed exteriorly of said housing and movable along a length thereof intermediate said proximal end and said distal end.

14. A handle assembly as recited in claim 11 wherein said positioning member and said stop member are concurrently movable a common distance relative to said housing and the tubular member respectively.

15. A. handle assembly as recited in claim 11 wherein said positioning member is removably disposable into any one of a plurality of locked positions on said body.

16. A handle assembly as recited in claim 1 wherein said restricting portion comprises a stop member extendable outwardly from said body along a length of said tubular member; said calibration assembly further comprising a positioning member at least partially defined by a calibration wheel rotationally connected to said body and disposed in driving relation to said stop member, said calibration wheel rotational on said body concurrently to movement of said stop member relative to said body.

17. A method facilitating the cleaning of an interior of an endotracheal tube utilizing a handle assembly having a hand held body structured to retain an expansion device and connected to a tubular member dimensioned to fit within and extend along a length of the endotracheal tube, said method comprising:
establishing a predetermined alignment between the tubular member and the endotracheal tube,
adjustably positioning a calibration assembly on the body in conformance with the alignment between the tubular member and the endotracheal tube,
inserting the tubular member within the endotracheal tube, and
disposing a stop member associated with the calibration assembly into confronting relation with an outer end of the endotracheal tube to determine an appropriate length of the tubular member being inserted within the endotracheal tube.

18. An assembly as recited in claim 17 comprising expanding a cleaning assembly mounted on the tubular member into a cleaning orientation relative to the endotracheal tube subsequent to the appropriate length of the tubular member being inserted into the endotracheal tube.

19. An assembly as recited in claim 18 comprising expanding the cleaning member by manipulating the expansion device retained on the body.

20. An assembly as recited in claim 18 comprising withdrawing the tubular member from within the endotracheal tube concurrently to maintaining the cleaning member in the cleaning orientation.
